(19) United States
(12) Patent Application Publication
(10) Pub. No.: US 2009/0264920 A1
(43) Pub. Date: Oct. 22, 2009

(54) CATHETER-BASED SEPTAL OCCLUSION DEVICE AND ADHESIVE DELIVERY SYSTEM

(76) Inventor: Alejandro BERENSTEIN, New York, NY (US)

Correspondence Address: William H. Dippert;Eckert Seamans Cherin & Mellott, LLC, U.S. Steel Tower, 600 Grant Street, 44th Floor, Pittsburgh, PA, 15219, US

(21) Appl. No.: 12/415,231

(22) Filed: Mar. 31, 2009

Related U.S. Application Data
(60) Provisional application No. 61/040,809, filed on Mar. 31, 2008.

(51) Int. Cl. A61B 17/03

(52) U.S. Cl. 606/213

(57) ABSTRACT

A catheter-based occlusion device for sealing tissue openings comprises a guiding catheter, a catheter, and a septal occluder including a first disk-like member for deployment proximate to a first side of a tissue opening, a second disk-like member for deployment proximate a second side of a tissue opening, and a connector portion for connecting the first and second disk-like members, wherein the connector portion includes at least one aperture effective to dispense an adhesive. The occlusion device can then be detached.

### CROSS-REFERENCE TO RELATED APPLICATION

This application is based upon and claims the benefit of the filing date of co-pending, commonly assigned U.S. Provisional Patent Application Ser. No. 61/040,809, filed Mar. 31, 2008, which is incorporated herein by reference in its entirety.

### FIELD OF THE INVENTION

The present invention is generally directed to a method and device used to close corporeal openings. More specifically, the invention is drawn to a catheter based occlusion device and adhesive delivery system for use in sealing openings in tissue, particularly, but not exclusively, in the heart.

### BACKGROUND OF THE INVENTION

Either congenitally or by acquisition, abnormal openings, holes, fistulae, or shunts can occur in human tissues, particularly between the chambers of the heart or in the vessels, causing blood to flow there through. Such deformities are typically congenital and originate during fetal development when the heart forms from a folded tube into a four chambered, two unit system. The deformities result from the incomplete formation of the septum or the muscular wall, between the chambers of the heart and can cause significant problems. The deformities can add strain on the heart, which may result in heart failure if they are not corrected. On other occasions it may permit the passage of clotted blood or foreign materials to the systemic circulation, that may then migrate toward other organs, such as the brain, and cause ischemic strokes.

One such deformity or defect, a patent foramen ovale (PFO), is a persistent, one-way, usually flap-like opening in the wall between the right atrium and left atrium of the heart. Since left atrial pressure is normally higher than right atrial pressure, the flap typically stays closed. Under certain conditions, however, if the hole is too big, and/or if the right atrial pressure exceeds the left atrial pressure, this creates the possibility for right to left shunting that can allow blood clots to enter the systemic circulation. This is particularly worrisome to patients who are prone to forming venous thrombus, such as those with deep vein thrombosis or clotting abnormalities.

Nonsurgical or percutaneous closure of PFO, as well as similar intercardiac defects such as atrial septal defects (ASDs), ventricular septal defects (VSDs), and left atrial appendages, is possible using a variety of mechanical closure devices.

### OBJECTS OF THE INVENTION

It is an object of the present invention to provide a closure apparatus and methods that overcome disadvantages of previous known devices.

It is also an object of the present invention to provide an apparatus and methods suitable for closure that quickly and effectively achieves hemostasis.

It further is an object of the present invention to provide apparatus and methods wherein all foreign materials left in a patient's body are not toxic to the patient.

It is yet another object of the present invention to provide a device effective to close septal openings such as patent foramen ovale.

It is still a further object of the present invention to provide a closure apparatus and methods that are safe, low cost and easy to use.

It is yet another object of the present invention to provide a device effective to close septal openings such as ASDs.

It is yet another object of the present invention to provide a device effective to close septal openings such as VSDs.

It is yet another object of the present invention to permit closure of defects of any size using microcatheter techniques for small access mostly when dealing with very small children.

These and other objects of the invention will become more apparent in the description below.

### SUMMARY OF THE INVENTION

According to the present invention, these and other of the above objects are attained by providing a septal occluder comprising a first disk-like member or mesh for deployment proximate to a first side of a tissue opening, a second disk-like member or mesh for deployment proximate a second side of a tissue opening, and a connector portion for connecting the first and second disk-like members. The connector portion includes at least one aperture effective to dispense an adhesive.

In another aspect of the invention, a catheter-based occlusion device for sealing tissue openings comprises a guiding catheter, a catheter, and a septal occluder including a first disk-like member for deployment proximate to a first side of a tissue opening and a second disk-like member for deployment proximate a second side of a tissue opening. A connector portion connects the first and second disk-like members. The connector portion includes at least one aperture effective to dispense a non-toxic, pharmacologically acceptable adhesive. The connector portion may be attached to or form a distal portion of the catheter, and the catheter may have one or more lumens, optionally one to dispense an adhesive.

A method of sealing tissues is also provided, comprising the steps of providing an apparatus comprising a guiding catheter, a catheter and at least two disk-like members, guiding the catheter to an opening and deploying an occluder comprising at least two disk-like members. The disk-like members are structured and arranged to expand from a compressed configuration to an expanded configuration, and the disk-like members are effective to seal the opening. An adhesive effective to seal the vascular opening is deployed, and the disk-like mesh constrains the liquid adhesive, on both sides, effectively sealing the hole on both sides. The two disk-like meshes and the adhesive are then left in place by a detachment mechanism.

Embodiments of the present invention are directed to devices for closing septal defects such as PFOs, and for delivering and recovering closure devices. The closure devices in these embodiments generally include a first disk-like member, a second disk-like member, and a connector portion for connecting the two disk-like members. The connector is a hypo tube, with at least a side hole, and may optionally have a second lumen for guidewire manipulation, fluid delivery, or contrast material injection. The disk-like mesh is made of nitinol, and may optionally incorporate a flexible elastomeric layer, which can also be used to promote tissue ingrowth or for drug delivery. The flexible material is also effective to dispense an adhesive to promote adherence to tissue or growth factors to accelerate tissue ingrowth.

In accordance with some embodiments of the present invention, options are provided for multiple delivery/recovery of the same device without withdrawing the device from the delivery sheath or otherwise replacing it. Other embodiments include the use of a single use delivery/recovery string that reduces the complexity of the delivery/recovery system and procedure itself.

According to one embodiment of the invention, a septal occluder comprises:
a first disk-like member for deployment proximate to a first side of a tissue opening;
a second disk-like member for deployment proximate a second side of a tissue opening; and
a connector portion for connecting the first and second disk-like members, wherein the connector portion includes at least one aperture effective to dispense an adhesive into said tissue opening.

According to another embodiment of a septal occluder of the invention, the tissue opening is a septal defect.

According to another embodiment of a septal occluder of the invention, the septal defect is a patent foramen ovale

According to another embodiment of the invention, a septal occluder further comprises a wire slidingly mounted in the first disk-like member, the wire being attached to the first disk-like member at one or more attachment points during delivery to the opening.

According to another embodiment of a septal occluder of the invention, at least one of the first disk-like member, the second disk-like member and the connector portion comprise a bioresorbable material.

According to another embodiment of a septal occluder of the invention, at least one of the first disk-like member, the second disk-like member and the connector portion comprise a non-bioresorbable material.

According to another embodiment of a septal occluder or catheter-based occlusion device of the invention, the adhesive is selected from the group consisting of cyanoacrylates, bioglues, tissue sealants and clotting agents.

According to another embodiment of a septal occluder or catheter-based occlusion device of the invention, the adhesive is bioabsorbable.

According to another embodiment of a septal occluder or catheter-based occlusion device of the invention, the adhesive is non-bioabsorbable.

According to another embodiment of the invention, a catheter based occlusion device for sealing tissue openings comprises:

a guiding catheter,

a delivery catheter; and

a septal occluder including

a first disk-like member for deployment proximate to a first side of a tissue opening;

a second disk-like member for deployment proximate a second side of a tissue opening; and

a connector portion for connecting the first and second disk-like members, wherein the connector portion includes at least one aperture effective to dispense an adhesive into said tissue opening

According to another embodiment of a catheter based occlusion device of the invention, the disk-like members are closely spaced together.

According to another embodiment of a catheter based occlusion device of the invention, the spacing between the disk-like members is capable of being adjusted.

According to another embodiment of a catheter based occlusion device of the invention, the disk-like members comprise nitinol mesh.

According to another embodiment of a catheter based occlusion device of the invention, the nitinol mesh further comprises a radiopaque marker.

According to another embodiment of a catheter based occlusion device of the invention, the nitinol mesh is lined with a polyurethane or similar material that enhances tissue growth.

According to another embodiment of a catheter based occlusion device of the invention, the septal occluder is detachable from the catheter.

According to another embodiment of a catheter based occlusion device of the invention, the adhesive is selected from the group consisting of cyanoacrylates, bioglues, tissue sealants and clotting agents.

According to another embodiment of a catheter based occlusion device of the invention, the adhesive is bioabsorbable.

According to another embodiment of a catheter based occlusion device of the invention, the adhesive is non-bioabsorbable.

According to another embodiment of the invention, a method of sealing tissues comprises the steps of:

providing an apparatus comprising a guiding catheter, a catheter and at least two disk-like members;

guiding the catheter to a tissue opening;

deploying a septal occluder comprising at least two disk-like members, the members structured and arranged to expand from a compressed configuration to an expanded configuration, the disk-like members effective to seal the tissue opening; and

dispensing an adhesive effective to seal the tissue opening.

According to another embodiment of the invention, a method of sealing tissues further comprises removing the apparatus from the patient.

According to another embodiment of a method of sealing tissues according to the invention, the tissue opening is a septal defect.

According to another embodiment of the invention, the connector is attached to and/or forms the distal portion of the catheter.

According to another embodiment of the invention, there is a frangible, breakable, or otherwise separable connection between the connector and the distal end of the catheter.

According to another embodiment of the invention, adhesive is dispensed through a lumen in the catheter.

According to another embodiment of the invention, the connector can be detached from the distal end of the catheter.

According to another embodiment of the invention, the septal occluder is arranged around and carried by the distal end of the catheter.

According to another embodiment of the invention, the connector comprises means to store and/or release adhesive.

According to another embodiment of the invention, adhesive is dispensed through or from the connector after activation.

These and other features and advantages of this invention are described in or are apparent from the following detailed description of exemplary embodiments.

### BRIEF DESCRIPTION OF THE DRAWINGS

A more complete appreciation of the present invention and many of the attendant advantages thereof will be readily understood by reference to the following detailed description of a preferred embodiment when taken in conjunction with the accompanying drawings, in which:

FIG. 1 is a substantially cross-sectional view of a catheter-based occlusion device and adhesive delivery system in accordance with an embodiment of the invention;

FIG. 2 is a lateral, substantially cross-sectional view of the catheter based occlusion device and adhesive delivery system in accordance with an embodiment of the invention;

FIG. 3 is a front perspective view of the catheter based occlusion device and adhesive delivery system of FIG. 1 in an undeployed position; and

FIG. 4 is a side perspective view of a septal occluder in accordance with an embodiment of the invention.

### DETAILED DESCRIPTION OF THE INVENTION

With reference to the drawings, in which like reference characters designate identical or corresponding parts throughout the several views, a catheter-based occlusion device and adhesive delivery system, generally designated 10, constructed in accordance with the present invention, is shown.

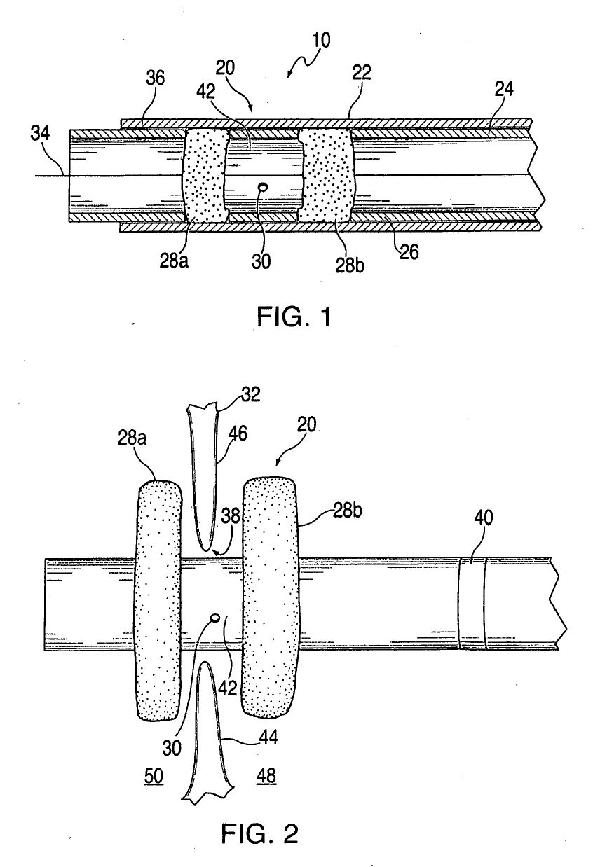

In an embodiment of the invention, a catheter-based occlusion device and adhesive delivery system is suitable for closing openings in a patient's heart (e.g., PFO, septal defects, etc.) or elsewhere in the body. As shown in FIGS. 1 to 3, a catheter-based occlusion device and adhesive delivery system comprises a guiding catheter 22, with a catheter 24 disposed therein, the catheter having at least one, preferably at least two, longitudinally extending lumens disposed within the body of catheter device 24. At the distal end 26 of catheter 24 a septal occluder 20 comprises at least two disk-like members 28a and 28b arranged circumferentially at distal end 26 of catheter 24. A connector portion 42 connects the disk-like members.

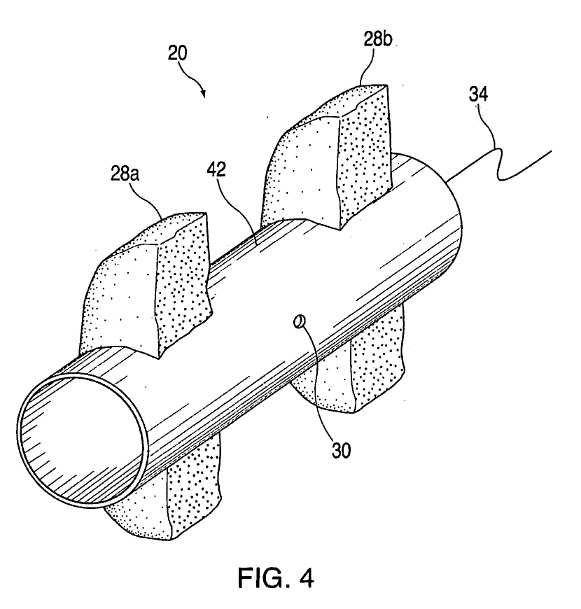

Disk-like members 28a, 28b are a nitinol or other metal mesh construction optionally with an additional polyurethane, foam silicon, or similar material closely spaced, and there is at least one opening 30 on the surface of connector portion 42 between disk-like members 28a, 28b, as shown in FIG. 4. The at least one opening 30 is effective to dispense an adhesive to a target tissue or site when deployed. The adhesive may be a tissue sealant, bioglue, or the like. When disk-like members 28a, 28b are disposed within guiding catheter 22, the disk-like members are in a first compressed position, having substantially the same outer diameter (OD) as catheter 24. When catheter 24 is deployed and guiding catheter 22 is withdrawn, disk-like members 28a, 28b expand to a second position, having a substantially larger diameter than that of catheter 24.

In one embodiment of the invention, anchor connector portion 42 and disk-like members 28a, 28b are made of a nonabsorbable and non-toxic material such as nitinol, and the connector portion 42 may be made of a suitable polymer or stainless steel. Disk-like members 28a, 28b may be covered with a material that can be fabricated from a single piece of bioresorbable polymer or by a laminated composite of two or more materials to provide a mix of properties. For example, disk-like members 28a, 28b can have stiff centers and flexible edges, and blood contacting surfaces can have controlled porosity or surface texture to promote fast and thorough endothelialization, while minimizing thrombosis. In addition, the tissue contacting surface of the disk-like members 28a, 28b can be designed to provide added stability, such as being roughened. The device is detached once in the desired position.

Guide catheter 22 and catheter 24 each comprise conventional physiologically acceptable materials that are known in the art. Optionally the distal end of catheter 24 may comprise material that is compatible with connector 42.

In another embodiment of the invention, anchor connector portion 42 and disk-like members 28a, 28b are bioresorbable and non-toxic. The materials can be fabricated from a single piece of bioresorbable polymer or by a laminated composite of two or more materials to provide a mix of properties. For example, disk-like members 28a, 28b can have stiff centers and flexible edges, and blood contacting surfaces can have controlled porosity or surface texture to promote fast and thorough endothelialization, while minimizing thrombosis. In addition, the tissue contacting surface of disk-like members 28a, 28b can be designed to provide added stability, such as being roughened. Other components, such as connection balls and strings, can also be made of bioresorbable materials, e.g., a string can be made of bioresorbable fibers that are braided or otherwise combined for strength.

In size, disk-like members 28a, 28b may be from about 2 mm to about 60 mm, preferably from 3 mm to about 50 mm, in diameter when fully expanded and from about 1.5 mm to about 8 mm, preferably from about 2 mm to about 6 mm, thick. In one embodiment one disk-like member may have a smaller diameter and/or thickness than the other disk-like member. Other structures are possible. For example, the sides of disk-like members 28a, 28b can be flattened, especially the side which will contact the atrial septum. While disk-like members 28a, 28b are generally circular in expanded form, disk-like members 28a, 28b may have some portions with different shaped cross-sections, with flattened sides, cut-outs, or other variations from an “ideal” circle. Disk-like members 28a, 28b can be elastomeric and resilient and made from a material, such as polyester, biological tissue, bioresorbable polymer, small diameter springs (e.g., nitinol), or spongy polymeric material, silicon foam, etc., and can include thrombogenic or inflammatory materials. Useful materials include, but are not limited to, elastomeric matrices and other materials disclosed in U.S. Patent Publication No. 2004/0260272, published Dec. 23, 2004, U.S. Patent Publication No. 2005/0043585, published Feb. 24, 2005, U.S. Patent Publication No. 2005/0043816, published Feb. 24, 2005, U.S. Patent Publication No. 2005/0165480, published Jul. 28, 2005, and U.S. Pat. Nos. 5,22,431, 5,382,259, 5,755,774, 5,823,198, 6,221,066, 6,299,619, 6,238,403, 6,280,457, 6,458,127, and 6,723,108, each of which is incorporated herein by reference in its entirety. Alternatively, disk-like members 28a, 28b can be made of multiple strands of materials such as polymer or metal, e.g., platinum, stainless steel, or nitinol, fibers. Disk-like members 28a, 28b can be textured or porous. These kinds of surfaces can also produce inflammatory responses, and therefore can promote faster tissue ingrowth and faster defect closure.

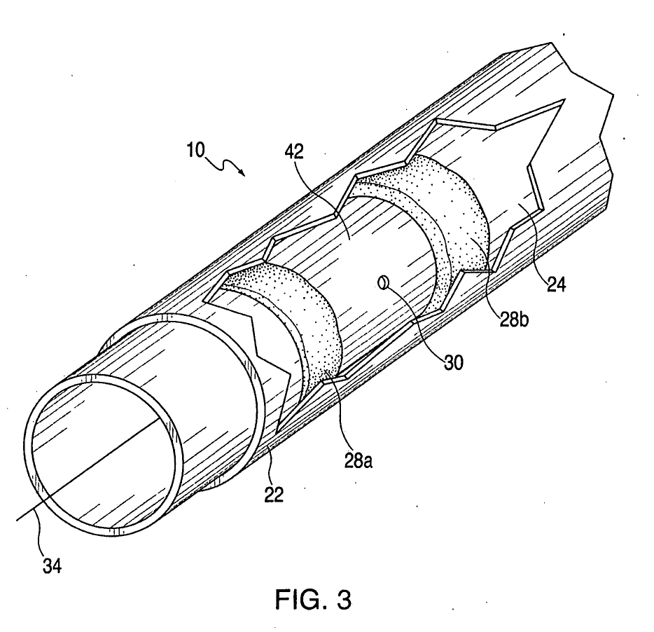

With reference to FIG. 2, catheter 24, disposed within guiding catheter 22, is directed to a desired site, such as a PFO or other tissue opening by guiding catheter 22, optionally over a guidewire 34. Catheter 24 is configured to provide maximum torque so that catheter 24 can be positioned properly at juncture/opening 38 of tissue 32. Prior to deployment first disk-like member 28a and second disk-like member 28b are compressed and are prevented from expanding by the inner surface of guiding catheter 22. Upon deployment past the distal end 36 of guiding catheter 22, first disk-like member 28a and second disk-like member 28b expand from a compressed first position to an expanded second position.

In one embodiment of the invention one or both disk-like members can be resheathed and replaced or repositioned if needed. In another embodiment of the invention, one or both disk-like members 28a, 28b can be repositioned and the distance between i.e., the connector portion 42, can be adjusted, if desired.

Catheter 24 is preferably guided to a position adjacent to juncture/opening 38 of tissue 32. Such an opening may include a PFO or a septal defect. Catheter 24 is structured and arranged to be received through juncture/opening 38. Catheter 24 is deployed such that first disk like member 28a expands on a first side 50 of a tissue opening, such as, for example, the left atrial side of a PFO, and second disk-like member 28b expands on a second side 48 of a tissue opening, such as, for example, the right atrial side of a PFO. The positioning is typically done by providing guiding catheter 22 in or at the edge of a left atrium on a first side 50 of tissue through opening 32. Guiding catheter 22 is retracted so that first disk like member 28a is deployed in the left atrium against a septum primum 44 and septum secondum 46. Guiding catheter 22 is further retracted until second disk-like member 28b is deployed in the right atrium. Connector portion 42 is disposed between septum primum 44 and septum secondum 46 and connects first disk-like member 28a and second disk-like member 28b.

Disposed on anchor connector portion 42 is at least one opening 30, which is preferably positioned at the juncture/opening 38. Upon completion of positioning of catheter 22 and disk-like members 28a and 28b, adhesive is deployed through at least one opening 30 into juncture/opening 38. The adhesive is effective to seal the juncture/opening 38. Contrast solution can be injected prior to deployment of the adhesive, or in another embodiment, contrast solution can be injected after deployment of the adhesive to detect any remaining leak. The adhesive may be a cyanoacrylate, bioglue, tissue sealant, clotting agent, or any other bioabsorbable adhesive. The adhesive can be made radiopaque, for example, with tantalum powder, gold colloid, or another radiopaque agent. The adhesive can be delivered to opening 30 through one or more lumens within catheter 24 or, optionally, adhesive could be stored in or near connector 42.

In an embodiment, after the adhesive is injected and sets effectively sealing the opening, catheter 24 can be detached at detachment portion 40, leaving septal occluder 20 positioned at tissue juncture/opening 38. Detachment portion 40 may be located at any portion of the catheter proximal to second disk-like member 28b. When detached, connector portion 42 and disk-like-members 28a and 28b remain at the vascular tree and the remainder of catheter 24 may be withdrawn. In another embodiment of the present invention, catheter 24 can be twisted and withdrawn, effectively detaching disk-like members 28a and 28b and connector portion 42 and leaving them at the tissue opening. The detachment portion 40 can be frangible, releasable, detachable, or otherwise separable by mechanical, chemical, or electrical means. Optionally there could be a mechanical joining such as threading or another releasable connector.

In another embodiment of the invention the septal occluder system could be positioned on and/or carried by the distal end of catheter 24. For example, connector 42 could be an annular structure on the outer surface of catheter 24.

In another embodiment, disk-like members 28a and 28b and connector portion 42 can be retrieved if a practitioner determines that the device is not sitting properly. To retrieve the device, guiding catheter 22 is placed proximal to second disk-like member 28b while catheter 24 is connected and septal occluder 20 is pulled by guide wire 34.

With reference to FIG. 1, guidewire 34 may remain connected to the device where detachment portion 40 breaks off, leaving a loose strand of wire. The is effective to pull first disk-like member 28a and second disk-like member 28b and connector portion 42, effectively causing the device to rotate and dislodge from juncture/opening 32. In this embodiment, because some of the attachment points have been broken, the device can be retrieved once, but would typically not be redeployed in this form. This embodiment does provide the ability to deliver and retrieve the device using a wire.

Unless defined otherwise, all technical and scientific terms used herein have the same meaning as commonly understood by one of ordinary skill in the art to which this invention pertains. Although any methods and materials similar or equivalent to those described herein can also be used in the practice or testing of the present invention, the preferred methods and materials are now described.

The invention has been described with reference to an embodiment that illustrates the principles of the invention and is not meant to limit the scope of the invention. Modifications and alterations may occur to others upon reading and understanding the preceding detailed description. It is intended that the scope of the invention be construed as including all modifications and alterations that may occur to others upon reading and understanding the preceding detailed description insofar as they come within the scope of the following claims or equivalents thereof. Various changes may be made without departing from the spirit and scope of the invention.

I claim:

1. A septal occluder comprising:
a first disk-like member for deployment proximate to a first side of a tissue opening;
a second disk-like member for deployment proximate a second side of a tissue opening; and
a connector portion for connecting the first and second disk-like members, wherein the connector portion includes at least one aperture effective to dispense an adhesive into said tissue opening.

2. The septal occluder of claim 1, wherein the tissue opening is a septal defect.

3. The septal occluder of claim 1, wherein the septal defect is a patent foramen ovale.

4. The septal occluder of claim 1, further comprising a wire slidingly mounted in the first disk-like member, the wire being attached to the first disk-like member at one or more attachment points during delivery to the opening.

5. The septal occluder of claim 1, wherein at least one of the first disk-like member, the second disk-like member and the connector portion comprise a bioresorbable material.

6. The septal occluder of claim 1, wherein at least one of the first disk-like member, the second disk-like member and the connector portion comprise a non-bioresorbable material.

7. The catheter based occlusion device according to claim 1, wherein the adhesive is selected from the group consisting of cyanoacrylates, bioglues, tissue sealants and clotting agents.

8. The catheter based occlusion device according to claim 1, wherein the adhesive is bioabsorbable.

9. The catheter based occlusion device according to claim 1, wherein the adhesive is non-bioabsorbable.

10. A catheter based occlusion device for sealing tissue openings, the device comprising:
a guiding catheter,
a delivery catheter; and
a septal occluder including
a first disk-like member for deployment proximate to a first side of a tissue opening;
a second disk-like member for deployment proximate a second side of a tissue opening; and
a connector portion for connecting the first and second disk-like members, wherein the connector portion includes at least one aperture effective to dispense an adhesive into said tissue opening.

11. The catheter based occlusion device according to claim 10, wherein the disk-like members are closely spaced together.

12. The catheter based occlusion device according to claim 11, wherein the spacing between the disk-like members is capable of being adjusted.

13. The catheter based occlusion device according to claim 10, wherein the disk-like members comprise nitinol mesh.

14. The catheter based occlusion device according to claim 13, wherein the nitinol mesh further comprises a radiopaque marker.

15. The catheter based occlusion device according to claim 14, wherein the nitinol mesh is lined with a polyurethane or similar material that enhances tissue growth.

16. The catheter based occlusion device according to claim 10, wherein said septal occluder is detachable from said catheter.

17. The catheter based occlusion device according to claim 10, wherein the adhesive is selected from the group consisting of cyanoacrylates, bioglues, tissue sealants and clotting agents.

18. The catheter based occlusion device according to claim 10, wherein the adhesive is bioabsorbable.

19. The catheter based occlusion device according to claim 10, wherein the adhesive is non-bioabsorbable.

20. A method of sealing tissues comprising the steps of:
providing an apparatus comprising a guiding catheter, a catheter and at least two disk-like members;
guiding the catheter to a tissue opening;
deploying a septal occluder comprising at least two disk-like members, the members structured and arranged to expand from a compressed configuration to an expanded configuration, the disk-like members effective to seal the tissue opening; and
dispensing an adhesive effective to seal the tissue opening.

21. The method according to claim 20, further comprising removing the apparatus from the patient.

22. The method according to claim 20, wherein the tissue opening is a septal defect.
